(19) United States
(12) Patent Application Publication
(10) Pub. No.: US 2020/0279630 A1
(43) Pub. Date: Sep. 3, 2020

(54) DRUG DISPENSER SYSTEMS AND METHODS THEREOF

(71) Applicant: Infinite Designs, LLC, Phoenix, AZ (US)

(72) Inventor: Adam Gersten, Phoenix, AZ (US)

(21) Appl. No.: 16/025,392

(22) Filed: Jul. 2, 2018

(51) Int. Cl. G16H 20/13; G16H 10/60; G05B 15/02

(52) U.S. Cl. CPC G16H 20/13; G05B 15/02; G16H 10/60

(57) ABSTRACT

A drug dispenser system may include receiving a request, by a controller, of dispensing a new dose of a medication for a user, wherein at least one dose of the medication is held inside a drug dispenser machine; conducting, by the controller, a safety check; indicating, by a display, whether the new dose of the medication is safe to be dispensed; and dispensing the medication from the drug dispenser machine if the safety check is passed.

### TECHNICAL FIELD

Aspects of this document relate generally to a drug dispenser system, and methods for dispensing medications that reduce risks for the user.

### BACKGROUND

A patient may be prescribed multiple medications, and each medication may need to be taken at different frequencies. Patients may find it difficult to take medications at a correct dosage and frequency. Further, medications may be additive and may create adverse reactions for the user, whether taken alone or together with other substances, including other medications. Taking medication, therefore, presents two major challenges for patients. One is to remember to take the medication at the prescribed intervals. This task is especially challenging for the elderly. The other challenge is not to overdose or abuse prescription drugs. A drug dispenser system may be used to address these challenges.

### SUMMARY

According to an aspect of the disclosure, a drug dispenser system may comprise a drug dispenser machine and a medication cartridge. The drug dispenser machine may comprise a controller, a user interface, a drive system, and a display. The controller may have a processor and a memory, the controller configured to conduct a safety check. The user interface may be configured to interact with a user. The drive system may be configured to couple to the controller. The display may be configured to display information sent from the controller. The medication cartridge may be releasably loaded onto the drug dispenser machine. The medication cartridge may include at least one pill chamber, at least one protective plate, and a label plug. The at least one protective plate may cover at least a majority of the at least one pill chamber. The label plug may be configured to couple to the at least one pill chamber. The drive system of the drug dispenser machine may be configured to move the label plug and, in turn, move the at least one pill chamber upon receipt of a command to dispense a medication sent from the controller when the safety check is passed.

Particular embodiments may comprise one or more of the following features. The safety check may comprise at least one of checking whether a minimum period of time between doses has elapsed, checking whether a request time of the safety check is outside an adversely-indicated window of another medication, and checking whether a user's medication reaction history indicates the medication is safe for the user. The drug dispenser machine may further comprise a read/write device configured to couple to the controller; the label plug of the medication cartridge may further comprise a communication device; and the read/write device and the communication device may be configured to communicate with each other. The drug dispenser system may further comprise an indicator configured to couple to the controller, wherein the controller is configured to send a command to set the indicator into a warning mode when the safety check is not passed. The medication cartridge may comprise a substantially circular foot print, the medication cartridge further comprising a carousel containing the at least one pill chamber. The drug dispenser system may further comprise a first medication cartridge and a second medication cartridge, wherein the first medication cartridge comprises a first medication; the second medication cartridge comprises a second medication; the drug dispenser machine may further comprise a turntable; and the first medication cartridge and the second medication cartridge may be disposed on the turntable. The drug dispenser system may further comprise a first layer of the medication cartridge and a second layer of the medication cartridge, the first layer disposed over the second layer. The label plug of the medication cartridge may further comprise a spoilage chemical reservoir, the label plug configured to deploy a spoilage chemical stored in the spoilage chemical reservoir into each of the at least one pill chamber of the medication cartridge when an auto-spoilage condition is met. The medication cartridge may be packaged by a manufacturer with a medication inside the at least one pill chamber, and the label plug of the medication cartridge is primed and inserted into the medication cartridge by the manufacturer.

According to an aspect, a drug dispenser system may comprise a drug dispenser machine. The drug dispenser machine may comprise a controller comprising a processor and a memory, a drive system configured to couple to the controller, and a display configured to display information sent from the controller. The drug dispenser machine may be configured to receive a medication cartridge, the controller of the drug dispenser machine configured to conduct a safety check upon receipt of a dispensing request from a user, and the drive system configured to move the medication cartridge upon receipt of a command to dispense a medication from the controller when the safety check is passed.

Particular embodiments may comprise one or more of the following features. The drug dispenser system may further comprise one or more medication cartridges releasably loaded onto the drug dispenser machine, the one or more medication cartridges comprising at least one pill chamber. The one or more medication cartridges may further comprise a spoilage chemical reservoir, the drug dispenser machine configured to deploy spoilage chemical stored in the spoilage chemical reservoir into each of the at least one pill chamber of the one or more medication cartridges when an auto-spoilage condition is met. The safety check may comprise at least one of checking whether a minimum period of time between doses has elapsed; checking whether a request time of the safety check is outside an adversely-indicated window of another medication; and checking whether the user's medication reaction history indicates the medication is safe for the user.

According to an aspect, a drug dispenser system may comprise receiving a request, by a controller, of dispensing a new dose of a medication for a user, wherein at least one dose of the medication is held inside a drug dispenser machine; conducting, by the controller, a safety check; indicating, by a display, whether the new dose of the medication is safe to be dispensed; and dispensing the medication from the drug dispenser machine if the safety check is passed.

Particular embodiments may comprise one or more of the following features. The safety check may comprise checking whether a minimum period of time between the new dose and an immediately-prior dose of the medication has elapsed or whether a request time of the safety check is outside an adversely-indicated window of another medication. The drug dispenser system may be configured to display an amount of time to wait before the new dose of the medication is dispensed if the minimum period of time between the new dose and the immediately-prior dose has not elapsed or if the request time of the safety check is within the adversely-indicated window of another medication. The safety check may comprise checking whether the user's medication reaction history indicates the medication is safe for the user. The drug dispenser system may be configured to set an indicator to a warning mode and display at least a portion of the user's medication reaction history that indicates the medication is unsafe for the user. The drug dispenser system may be configured to check whether the medication has expired or whether a request time of the safety check is after a prescribed time of the medication. The drug dispenser system may be configured to start an auto-spoilage mechanism if the medication has expired or if the request time of the safety check is after the prescribed time of the medication.

Aspects and applications of the disclosure presented here are described below in the drawings and detailed description. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain, and ordinary meaning to the terms be applied to the interpretation of the specification and claims.

The inventors are also aware of the normal precepts of English grammar. Thus, if a noun, term, or phrase is intended to be further characterized, specified, or narrowed in some way, such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112(f). Thus, the use of the words “function,” “means” or “step” in the Detailed Description or Description of the Drawings or claims is not intended to somehow indicate a desire to invoke the special provisions of 35 U.S.C. § 112(f), to define the invention. To the contrary, if the provisions of 35 U.S.C. § 112(f) are sought to be invoked to define the inventions, the claims will specifically and expressly state the exact phrases “means for” or “step for”, and will also recite the word “function” (i.e., will state “means for performing the function of [insert function]”), without also reciting in such phrases any structure, material, or acts in support of the function. Thus, even when the claims recite a “means for performing the function of . . . ” or “step for performing the function of . . . ,” if the claims also recite any structure, material, or acts in support of that means or step, or to perform the recited function, it is the clear intention of the inventors not to invoke the provisions of 35 U.S.C. § 112112(f). Moreover, even if the provisions of 35 U.S.C. § 112(f), are invoked to define the claimed aspects, it is intended that these aspects not be limited only to the specific structure, material, or acts that are described in the preferred embodiments, but in addition, include any and all structures, material, or acts that perform the claimed function as described in alternative embodiments or forms in the disclosure, or that are well-known present or later-developed, equivalent structures, material, or acts for performing the claimed function.

The foregoing and other aspects, features, and advantages will be apparent to those artisans of ordinary skill in the art from the DETAILED DESCRIPTION and DRAWINGS, and from the CLAIMS.

### BRIEF DESCRIPTION OF THE DRAWINGS

Implementations will hereinafter be described in conjunction with the appended drawings, where like designations denote like elements.

FIG. 1A is a schematic diagram of a drug dispenser system.

FIG. 1B shows an example drug dispenser system depicted in FIG. 1A.

FIG. 2 is a flow chart of a safety check.

FIG. 3 is a flow chart of using a drug dispenser system.

FIG. 4A shows a drug dispenser system configured to receive more than one medication cartridge.

FIG. 4B shows the drug dispenser system of FIG. 4A without a cover and with a medication cartridge.

FIG. 5 is a flow chart of using the drug dispenser system shown in FIG. 4A.

FIG. 6 shows an example of a medication cartridge.

FIG. 7A shows a top view of the medication cartridge of FIG. 6 with a top protective plate removed.

FIG. 7B is a top view of another medication cartridge with its top protective plate removed.

FIG. 7C is cross-section view of the medication cartridge of FIG. 6.

FIG. 7D shows the label plug of the medication cartridge of FIG. 6.

FIG. 8A shows an example label plug before being inserted into a medication cartridge.

FIG. 8B shows the label plug of FIG. 8A inserted into a medication cartridge with the sleeve of the label plug being removed.

FIG. 9A shows the top portion of the label plug shown in FIG. 7D.

FIG. 9B shows a top view of the medication cartridge of FIG. 6 before medication has being dispensed.

FIG. 9C shows a top portion of the label plug of FIG. 7D with the catching device cover retracted.

FIG. 9D shows a drive system and a read/write device of the drug dispenser system to be engaged with the label plug of FIG. 6.

FIG. 9E shows a top view of the medication cartridge of FIG. 6, when the medication is being dispensed, with the top protective plate of the medication cartridge removed.

FIG. 9F shows the top portion of the label plug of FIG. 7D with the catching device returned to the pre-dispensing state.

FIG. 10A shows a multi-layered medication cartridge.

FIG. 10B shows the label plug of the medication cartridge of FIG. 10A.

FIG. 10C shows a drive system to be engaged with the first layer of the medication cartridge of FIG. 10A.

FIG. 10D shows the drive system to be engaged with the second layer of the medication cartridge of FIG. 10A.

### DETAILED DESCRIPTION

This disclosure, its aspects and implementations, are not limited to the specific material types, system component examples, or methods disclosed herein. Many additional components, manufacturing and assembly procedures known in the art consistent with product design and manufacture are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented, but have been omitted for purposes of brevity.

While this disclosure includes embodiments in many different forms, they are shown in the drawings and will herein be described in detailed particular embodiments with the understanding that the present disclosure is to be considered as an exemplification of the principles of the disclosed methods and systems, and is not intended to limit the broad aspect of the disclosed concepts to the embodiments illustrated.

A conventional drug dispenser system dispenses medications for a patient when a button is pressed or a handle is moved. Conventional drug dispenser systems do not check whether the medication is safe to be dispensed at the moment when the medication is being taken too close together with the medication itself or with another medication. Conventional drug dispenser systems also do not restrict hoarded or unused medication from getting into wrong hands and being overdosed or misused.

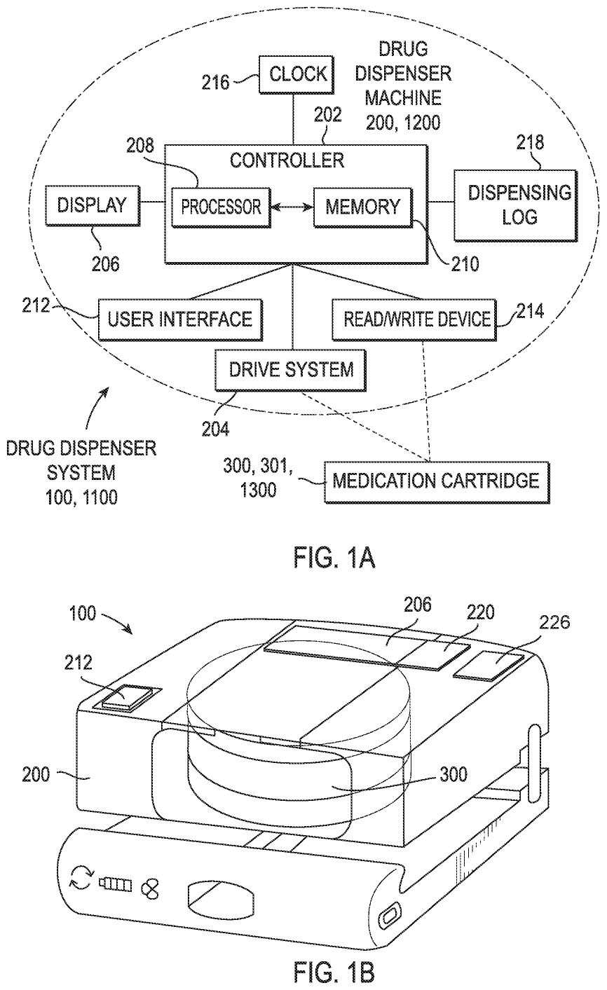

Contemplated as part of this disclosure are drug dispenser systems and methods of dispensing medications to reduce risks from the medications for the users. FIG. 1A shows a schematic diagram of the drug dispenser system 100, 1100. FIG. 1B shows an example drug dispenser system 100.

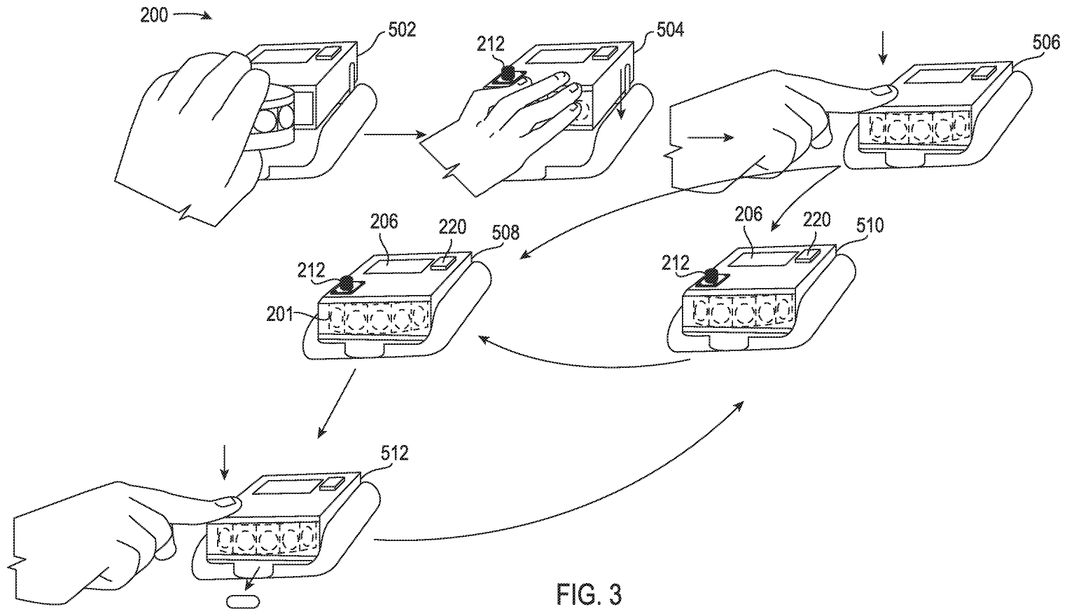

A drug dispenser system 100, 1100 comprises a drug dispenser machine 200, 1200 and a medication cartridge 300, 301, 1300. The drug dispenser machine 200 may further comprise a door 201 such that the drug dispenser machine 200 encloses the medication cartridge 300, 301, 1300 (FIG. 3). In some embodiments, the drug dispenser machine 200 does not include a door (FIG. 1B).

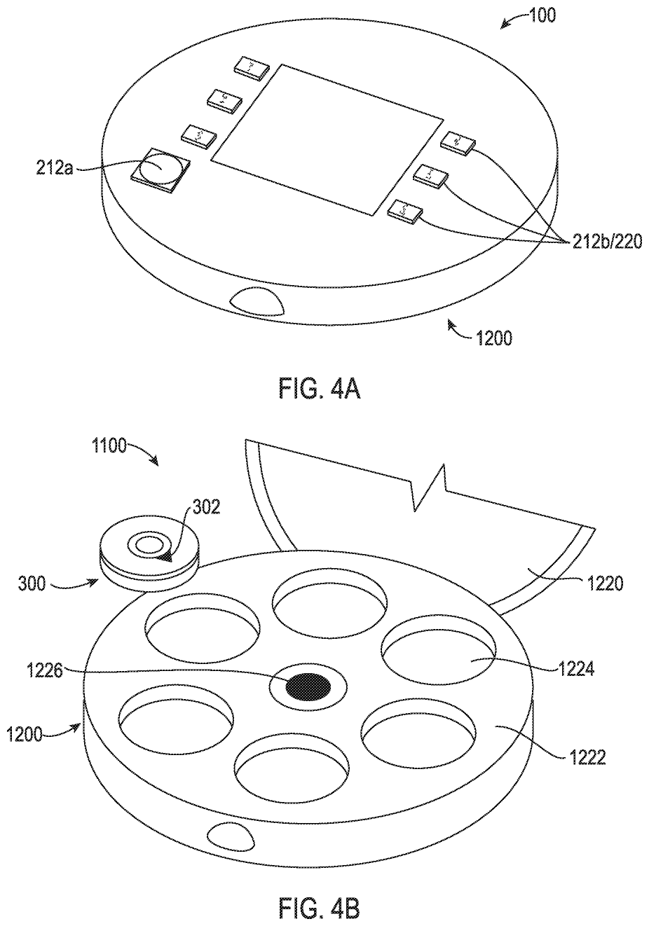

The drug dispenser machine 200, 1200 comprises a controller 202, a drive system 204, and a display 206. The controller 202 may comprise a processor 208 and a memory 210. The controller 202 is the control center of the drug dispenser machine 200, 1200. It may issue command and send and receive data, and may also start, run, and control processes run on the drug dispenser system 100, 1100. The drive system 204 may be coupled to the controller 202 and be used to drive the medication cartridge 300, 301, 1300. The display 206 may be used to display information to be seen by a user. The display 206 may be in Braille for visually-impaired users to read the information. The drug dispenser machine 200, 1200 may further comprise an indicator 220 to indicate the status of a dispensing process (FIGS. 1B, 3 and 4A). The indicator 220 may be part of the display 206 (FIG. 1B) or may be separate from the display 206 (FIGS. 3 and 4A). The drug dispenser machine 200, 1200 may further comprise a speaker to read out the information. The medication cartridge 300, 301, 1300 may be releasably loaded into the drug dispenser machine 200, 1200 or may be integrated into the drug dispenser machine 200, 1200 such that the drug dispenser system 100, 1100 is for one-time use. Multiple medication cartridges 300, 301, 1300 may be loaded to a drug dispenser machine 200, 1200.

The drug dispenser system 100 may further comprise data storage or storage. The storage may be in the drug dispenser machine 200, 1200, or may be an external storage such as the cloud. The drug dispenser machine 200, 1200 may further comprise a user interface 212, a read/write device 214, a clock 216, and a dispensing log 218. The user interface 212 is configured to interact with a user. The read/write device 214 may be used to read and/or write data to and from the controller and to and from the medication cartridge 300, 301, 1300. The clock 216 may be used to keep track of and control the timing of the drug dispenser system 100, 1100. The clock may be, and in some instances will be, radio-linked or otherwise coupled such that power loss or interruptions in the power supply will not stop, thwart, or limit, function or integrity of the system 100. As such, the time on the clock may be, and in some cases will be, automatically updated, regardless of whether the clock is connected to a power source. The dispensing log 218 may store dispensing data of the drug dispenser system 100, 1100, and may be stored in the memory 210 or the storage. The drug dispenser system 1000 may further comprise a size or dimensions in ranges of 15-50 centimeters (cm) long, 10-45 cm wide, and 1.5-40 cm thick, or other suitable dimensions.

Unlike conventional drug dispenser systems, which dispense medications at a time triggered by a timer or when requested, the drug dispenser system disclosed herein conducts safety checks before medications are dispensed. The drug dispenser system 100, 1100 is configured to check whether two doses of a medication are too close together, or whether the time taking one medication is too close to a time of taking another medication. The drug dispenser system 100, 1100 is also configured to check whether dispensing the medication is safe according to the user's allergy or medication reaction history stored or downloaded onto the system. The safety check may comprise checking whether a minimum period of time between doses has elapsed, checking whether the request time of the safety check is outside the adversely-indicated window of another medication, and checking whether the user's medication reaction history indicates the medication is safe for the user to take at the request time.

In some embodiments, the drug dispenser system 100, 1100 may comprise an anti-tampering feature, and the safety check further comprises an auto-spoilage check. The anti-tampering mechanism may be deployed when auto-spoilage conditions are met, for example, when the system has been tampered with, or when the medication is expired, or when the prescribed time of the medication has passed. The expiration time of a medication is often marked as “exp” or “used by.” The prescribed time of a medication is the duration of time that the doctor has prescribed for the patient to take the medication. For example, a doctor may prescribe a pain killer, asking the patient to take the pain killer within the prescribed time such as within the next week. In this example, the prescribed time would end at the end of the next week. A user should not take the medication after the expiration time or the prescribed time. The auto-spoilage check may comprise checking whether the medication loaded in the medication cartridge has expired, and may also include checking whether the request time for the safety check is after the prescribed time of the medication.

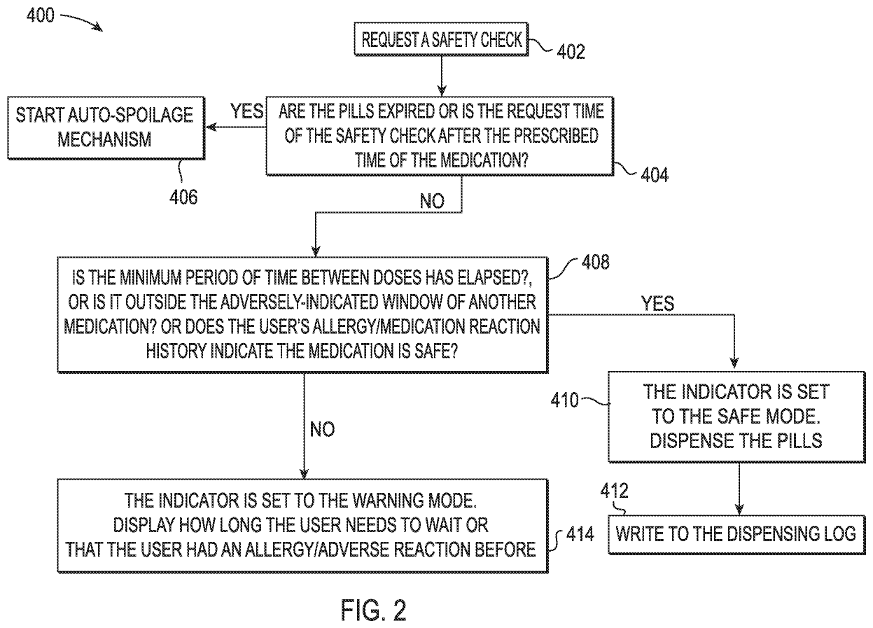

FIG. 2 shows a flow chart of an example process 400 of a safety check conducted by the drug dispenser system. A user may request medication be dispensed, or request a dose availability check, which will initiate a safety check, through the user interface. The user interface may relay the request for a safety check to the controller or trigger a request for a safety check upon a dispensing request. Upon the receipt of the request for a safety check, the controller may start the process of a safety check (402). The controller may check whether the condition for auto-spoilage is met (404). The controller may check whether the medications in the medication cartridge are expired (404). The controller may check whether the request time of the safety check is after the prescribed time for the medication (404). If the condition for auto-spoilage has been met (e.g., the medication has expired or the request time is after the prescribed time), the dispenser system starts the auto-spoilage mechanism to render the medication in the medication cartridge unusable or too unpalatable to be taken (406). In some embodiments, the safety check does not include an auto-spoilage check and, when a safety check is requested (402), the drug dispenser system 100, 1100 directly starts a regular safety check (408).

The controller may check whether the minimum period of time between doses has elapsed, whether the request is outside the adversely-indicated window of another medication, or whether the user's allergy/medication reaction history indicates the medication is safe for the user to take (408). If the answers to all of the questions are yes, the indicator is set to the safe mode and the medication is dispensed (410). The read/write device may write to the dispensing log in the drug dispenser system (412). If the answer to any of the question is no, the indicator is set to the warning mode and the display may display how long the user needs to wait until the medication may be dispensed or display that the user had an allergy or adverse medication reaction in the past (414).

The dispensing log may comprise two logs, one for the safety check and one for record keeping. The dispensing log may be stored on the dispensing machine or on the communication device of the label plug. In some embodiments, the dispensing log for safety check may include only the last 24-hour dispensing data of the drug dispenser system. The duration of 24 hours is suitable for examining the drug interaction with the same medication or a different medication because acute adverse effects are most likely to be visible during the next 24 hours after the consumption of the medication. It may also be suitable for patients' privacy or security concerns that data not be transmitted wirelessly, and not be available to be forcibly extracted or disclosed without proper authorization or under improper circumstances. The log may be overwritten or cleared every twenty-four hours, or at any other suitable fixed period of time. The record keeping log may comprise basic information such as whether a medication has taken on a certain day so that the system can keep a record. The clock of the drug dispenser system may be used to control and keep track of the duration of the dispensing log.

In some instances, a lock-out code may be programmed onto a medication disc's microchip to lock-out prospective dispensing of medication from other medication discs based upon a superseding prescribing/dispensing date of newer prescriptions of the same medication, medications within the same class, or for other reasons, such as law enforcement-related, reasons. The lock-out command could be written from the dispenser device to each new medication disc with that same identification, such as for a same patient's name, and thereby propagate itself to all medication discs associated with that patient's name that have contact with a dispenser device that has received the lock-out command, either directly or indirectly, such as through medication disks. As such, any subsequent prescriptions (of any medication dispensed via Medication Discs) may serve to prevent drug abuse by locking out future dispensing from a common dispenser device. In some instances, the lock-out command could even be stored to a “secure enclave” of the dispenser's memory and be maintained for more than 24-hours or other security/privacy type memory restriction. The lock-out command may even serve to activate the auto-spoilage mechanism upon the first insertion of a verboten medication disc into the dispenser device that has received the lock-out command. As such, propagation of a lock-out command may be securely communicated without unreasonably endangering patient privacy/security.

While the above approach may be available for devices that are restricted from wireless communications, such as for security/privacy reasons, internet connected dispenser devices, may receive internet or wireless enabled commands or stop orders.

As mentioned herein, in some instances a dispenser device will not keep any memory/log beyond a certain period, such as 24-hours, to limit exposure and liability to release of medical information from the memory/log. In other instances, the dispenser device may keep a memory/log for longer than 24-hours, such as for lock-out codes, or only for lock-out codes, associated with given individuals, the memory/log being stored to a permanent (or at least long-duration) memory on the dispenser device itself. Then, when a new medication disc is inserted into the dispenser device, the dispenser device will read the new disc, check the patient's identity against any lock-out codes stored on the dispenser's “Secure Enclave” and only if there is a code associated with that patient present in memory will the dispenser device check those code(s) against the medication disc to see if a lock-out of that medication, that class of medications, or all controlled substance prescriptions are in effect.

FIG. 3 shows a flow chart of a user using a drug dispenser system. The user pushes a medication cartridge into a drug dispenser machine (502). The medication cartridge engages with the drug dispenser machine (504). The user presses the user interface 212, e.g., a button, requesting to dispense a medication and the drug dispenser machine conducts a safety check (506). Depending on the result of the safety check, the drug dispenser machine indicates that the medication is safe to be dispensed (508) or unsafe to be dispensed (510). If the safety check is passed, the drug dispenser system may set the indicator 220 to a safe mode, e.g., setting the indicator 220 to green, indicating the medication is safe to be dispensed and start the dispensing process (508). If the safety check is not passed, the drug dispenser machine may indicate the medication is unsafe to be taken at the request time by setting the indicator 220 at a warning mode, e.g. setting the indicator to red (510). In some embodiments, the drug dispenser machine may display through the display 206 how long the user needs to wait before the medication is dispensed. Once the wait time has elapsed, the drug dispenser machine may start the dispensing process 508, which may occurs after, or only after, a user requests dispensing of the medication, for example. Both elapsed wait time and a user request may prevent automatic or undesired dispensing when no user is present or ready for medication. In some embodiments, the drug dispenser machine may indicate the medication unsafe to be dispensed at the request time if the user had an allergy or an adverse reaction to the medication in the past. The drug dispenser machine may display, through the display 206, information about the allergy or the adverse reaction. After the drug dispenser system starts the dispensing process, the medication is dispensed (512). The next time when the user requests the medication to be dispensed (506), the safety check process (506-512) starts again.

In some embodiments, the drug dispenser machine is configured to receive more than one medication cartridges. FIGS. 4A and 4B show an example drug dispenser system 1100 comprising a drug dispenser machine 1200 configured to receive multiple medication cartridges 300. The medication cartridges may be loaded with the same or different medications. FIG. 4A shows an example drug dispenser system 1100. FIG. 4B shows the example drug dispenser system 1100 with the cover 1220 of the drug dispenser machine 1200 removed to shown the inside of the drug dispenser machine 1200. The drug dispenser machine 1200 comprises a turntable 1222. The turntable 1222 comprises one or more cartridge slots 1224 configured to receive medication cartridges 300. The medication cartridge 300 may comprise a placement marker 302. The drug dispenser machine 1200 may comprise a centering marker 1226 disposed on the center of the turntable 1222. In loading medication cartridges 300 into the drug dispenser machine 1200, the placement marker 302 is aligned with the centering marker 1226 so that the medication cartridges all start at the same starting points. In the example drug dispenser system 1100, shown in FIGS. 4A and 4B, multiple cartridge slots 1224 (such as six cartridge slots) are provided and up to six medication cartridges 300 may be loaded onto the system. A person of ordinary skill in the art will appreciate that any desirable number of cartridges may be used, such as 1, 2, 3, 4, 5, or more. In some instances, cartridges will be of a same uniform size, and in other instances they may vary in size. In some instances, the multiple cartridges may be arranged in a turntable configuration, as illustrated for example in FIG. 4B. In other instances, the cartridges may be vertically stacked one above another, such as with a front-loading arrangement, and may be similar to the arrangement shown in FIG. 1B.

A user can request, through the user interfaces 212, the medications in the medication cartridges be dispensed. In some embodiments, the drug dispenser system 1100 may comprise a user interface 212b for each medication cartridge, or a single interface for more than one medication cartridge, including all the cartridges. The user interface 212b may be combined with the indicator 220 so that, when a user presses on a user interface 212b for a specific medication cartridge, the user interface 212b/indicator 220 indicates whether the medication in the specific medication cartridge is safe to be dispensed at the request time. In some embodiments, the drug dispenser system may comprise a user interface 212a that allow the user to inquire the safety of the medications in all medication cartridges at the same time. In some embodiments, the user interface allows the user to inquire about subsets of issues of the medications using subsets of the user interface.

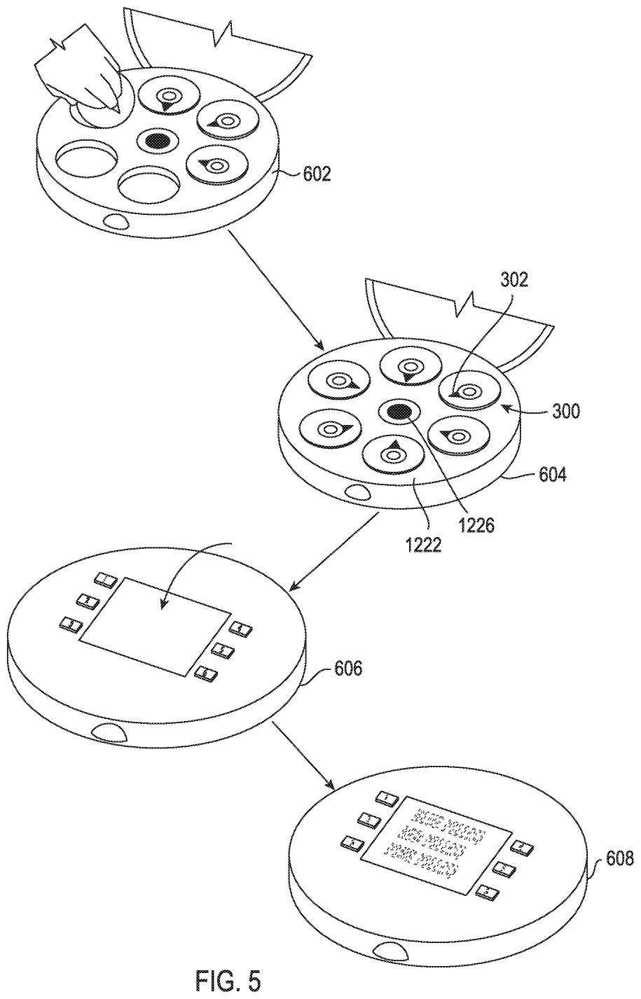

FIG. 5 shows an example flow chart of using a multi-cartridge dispenser system. First, multiple cartridges are loaded onto the drug dispenser machine (602). In loading the medication cartridges, the medication cartridges are aligned (604). In aligning the medication cartridges 300, the placement marker 302 on each medication cartridge 300 aligns to the centering marker 1226 on the turntable 1222 such that the medication cartridges all start at the same points. Once the cartridges are loaded, the drug dispenser system is ready for use by a user (606). A user may inquire about the medications in the medication cartridges and the medications may be dispensed (608). Because each medication cartridge 300 may be individually dispensed, the drug dispenser machine 1200 may be loaded with medication cartridges 300 having different medications, especially medications with different dosage frequencies or medications having different adversely-indicated windows with themselves or other medications.

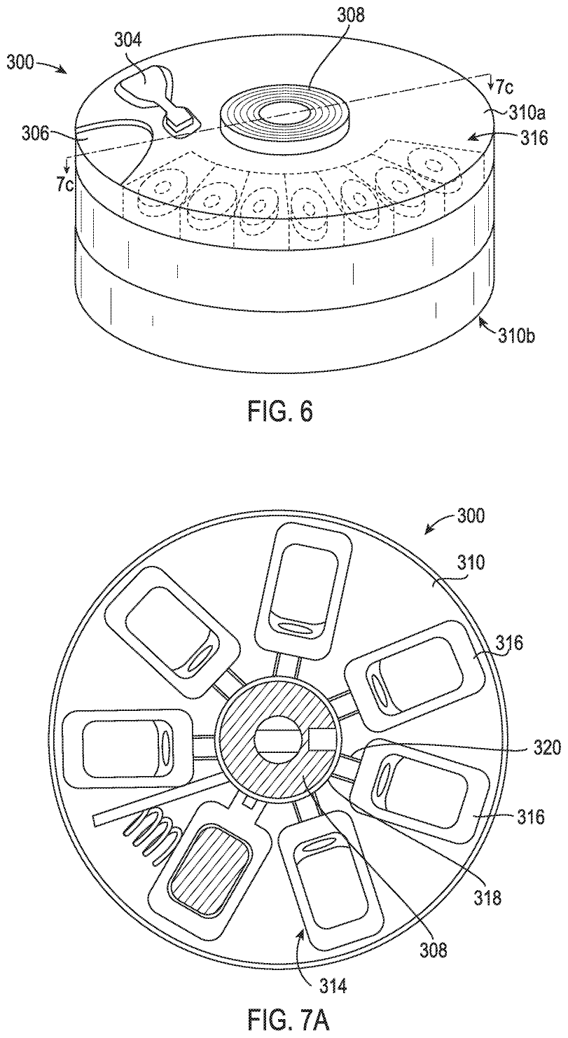

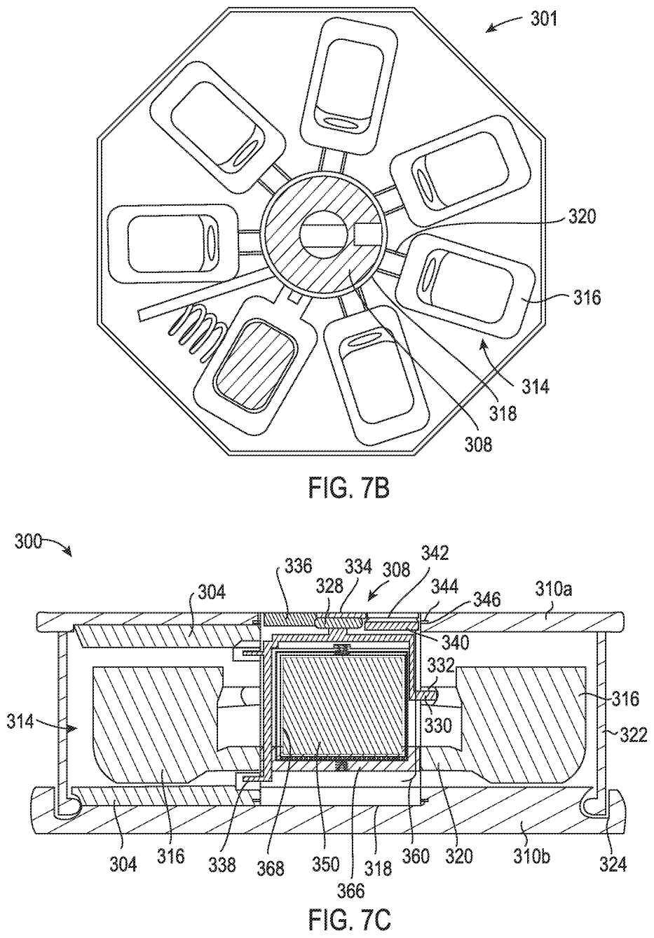

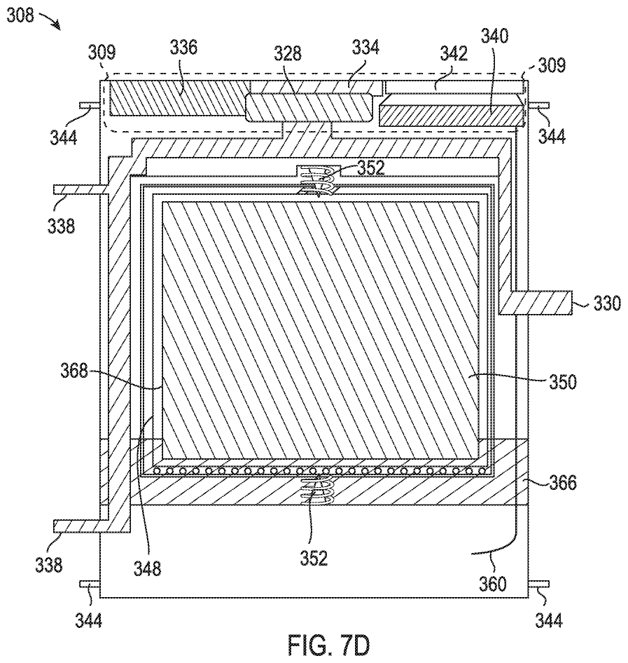

An example medication cartridge 300 is shown in FIG. 6 with the pill cover 304 pulled away from the pill opening 306 for the purpose of illustration. FIGS. 7A-7D show various views and parts of a medication cartridge 300, 301. FIG. 7A and FIG. 7B show a medication cartridge 300, 301 with the top protective plate 310a removed. FIG. 7C shows a cross-section view of the medication cartridge 300. FIG. 7 shows a label plug 308 by itself

The foot print of the medication cartridge 300, 301 may be circular or polygonal (FIG. 7B). The foot print may be substantially circular such that the foot print of the medication cartridge 300, 301 roughly follows a circle (FIGS. 6, 7A, and 7B). When the foot print of the medication cartridge is a curve, the foot print is substantially circular when the distances between points on the foot print and corresponding points on the closest circle to the foot print are in the range of 0-5%, 2-10%, or 1-20% of the radius of the closest circle. If the foot print is polygonal, 75-100%, or 60-100%, or 85-100% of the vertexes of the polygon formed by the foot print are on the closest circle of the foot print. The medication cartridge may be in other shapes, such as cube, cuboid, cylinder, or prism. The size of a medication cartridge may be in a range of 15-45 cm long, 10-40 cm wide, and 1.5-35 cm thick, or of any other suitable dimension according to a number and size of pills to be dispensed. While the description describes medication doses with respect to pills or capsules, other forms of disbursing appropriate doses are also contemplated herein, such as dosages dispensed as liquids or syrups.

The medication cartridge 300, 301, 1300 may comprise a label plug 308, 1308, at least one pill chamber 316, and at least one protective plate 310 that covers a majority of the at least one pill chamber 316 (FIGS. 6-10B). The label plug 308, 1308 is configured to couple to and drive the pill chambers 316. When the safety check is passed, the controller may send out a dispending command and control the drive system to engage with and drive the label plug and, in turn, the label plug may move the pill chambers to dispense medications. Medication cartridges may be formed of plastic, metal, wood, or other material known to a person skilled in the art.

The protective plate or part of the protective plate may be translucent so that a user may see through the protective plate to check if the medication cartridge has any medication left. The medication cartridge 300, 301, 1300 may further comprise a side 322 (FIGS. 7A-7D). The side 322 may be tension fitted into the recess 324 on the bottom protective plate 310b. The protective plate 310 may comprise a pill opening 306. The medication cartridge 300, 301, 1300 may further comprise a pill cover 304. The pill cover 304 may cover the pill opening 306. In some embodiments, the medication cartridge may comprise a top protective plate 310a and a bottom protective plate 310b to cover the medication loaded inside (FIGS. 6 and 7C). A medication cartridge having both a top protective plate and a bottom protective plate may be convenient for a multi-layered medication cartridge configuration, where one medication cartridge may be placed on top of another and, during dispensing of medications, all of the pill openings on the protective plates are lined up so that the medication on the top layer may be dispensed through the pill openings.

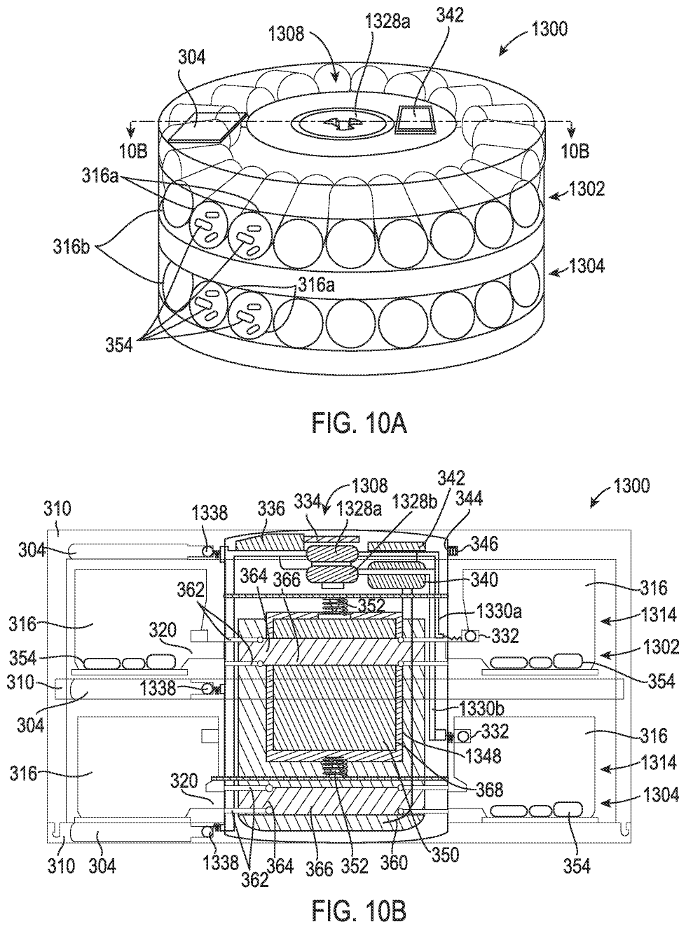

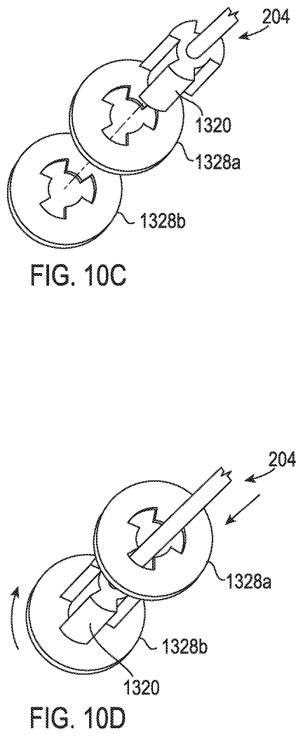

The medication cartridge 300, 301, 1300 may comprise a carousel 314, 1314 (FIGS. 7A-7C, and 10B). A carousel 314, 1314 comprises one or more pill chambers 316. The pill chambers may be arranged around a hub 318. In some embodiments, each pill chamber 316 is connected to the hub through a channel 320. The channel may be used to release spoilage chemical into the pill chambers when the medication cartridge is tampered or the auto-spoilage mechanism is deployed.

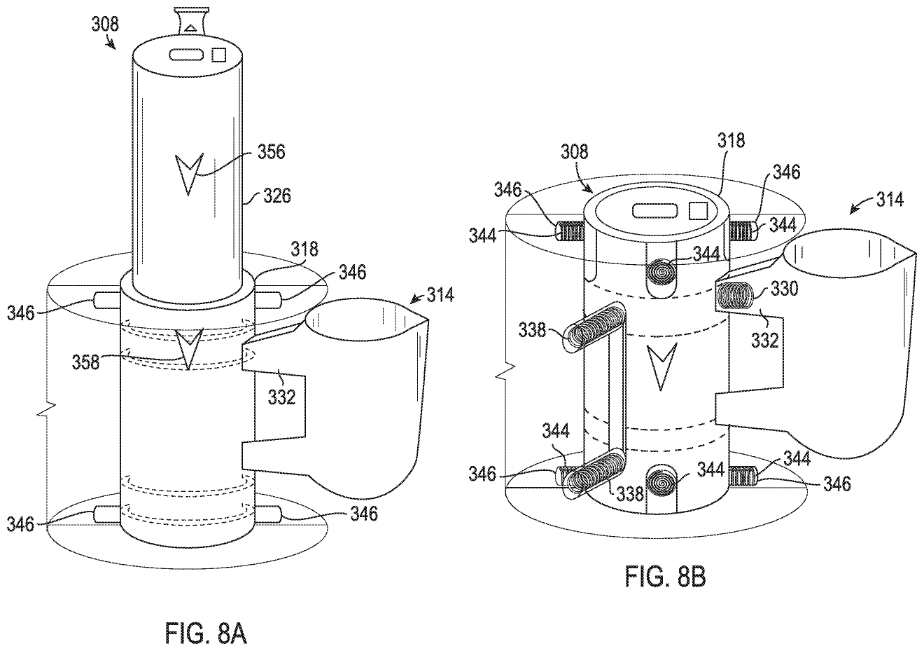

A label plug 308, 1308 may be inserted into the medication cartridge 300, 301, 1300. In some embodiments, the label plug 308, 1308 is inserted into the hub 318 of the carousel 314, 1314 (FIGS. 7A, 7B, 7C, and 10B). A label plug is used to drive the carousel and may also be used to deploy the auto-spoilage mechanism. FIGS. 7D and 10B show an example label plug 308, 1308 with its sleeve 326 removed (an example sleeve 326 is shown in FIG. 8A).

The label plug 308, 1308 may comprise one or more locking bars 344 disposed on the sides of the label plug 308, 1308. The locking bars 344 are used to secure the label plug into the hub 318 of the carousel 314, 1314 by engaging into the cover recesses 346 in the protective plates 310. A label plug 308 may comprise a catching device 328 configured to be driven by the drive system 204 of the drug dispenser machine 200, 1200, and one or more drive arm 330, 1330a, 1330b coupled to the catching device 328. Each of the drive arm 330, 1330a, 1330b may engage with a recess 332 on a pill chamber 316 so that, when the drive system 204 engages with the catching device 328, the drive system 204 drives the pill chamber 316 through the drive arms 330, 1330a, 1330b. The label plug 308, 1308 may further comprise a catching device cover 334 and a cover actuator 336. The catching device cover 334 may be used to cover the catching device 328 to restrict the catching device 328 from being engaged with the drive system 204. The cover actuator 336 may control the movement of the catching device cover 334. A non-limiting embodiment of a cover actuator may be a solenoid that magnetically attracts, or loses the attraction of, the catching device cover when electric current is turned on or off.

The label plug 308, 1308 may further comprise a pill cover activating bar 338, 1338 which may push the pill cover 304 away from the pill opening 306 so that medications may be dispensed through the pill opening 306. The label plug 308, 1308 may further comprise a communication device 340. The communication device 340 may, and in some cases will, be a radio-frequency identification chip, a microchip, or other devices that contains electronically stored information. The communication device 340 may comprise dispensing data. By comprising a radio-link or other suitable connection, the communication device 340 may continue to function without interruption, and without a loss of system integrity, in the case of power loss or power interruptions interruption. The label plug 308, 1308 may further comprise a read/write device connector 342. The communication device 340 is operably coupled to the read/write device 214 of the drug dispenser machine 200, 1200. The coupling between the communication device 340 and the read/write device 214 may be through the read/write device connector 342. The communication device 340 may directly communicate with the read/write device 214. For example, in some instances the read/write device 214 may directly read or write from the communication device 340 through a direct connection and not wirelessly, to prevent hacking or security compromises to the medical/dispensing history. In other instances, communication may be done wirelessly.

The label plug 308, 1308 may further comprise a spoilage chemical reservoir 348, 1348 stored with spoilage chemical 350 (FIGS. 7C, 7D, and 10B). In some embodiments, the spoilage chemical may be light sensitive such that, if the medication cartridge is tampered and light enters inside the medication cartridge, the spoilage chemical is activated. The spoilage chemical 350 may be contained in a membrane 368. The membrane may be formed of plastics. The label plug 308, 1308 may further comprise one or more pins 352. When auto-spoilage mechanism is deployed either because the auto-spoilage check has failed or because the drug dispenser machine 200, 1200 is tampered, the pins 352 are pushed toward and puncture the membrane 368. As a result, the spoilage chemical 350 flows through the trough 366 and through the channel 320 into each pill chamber 316 to render the medications useless. The spoilage chemical may neutralize or sequester the active ingredients in the medication. The spoilage chemical may render the medication so unpalatable that a person would not take the medication. The spoilage chemical may be a chemical heater and incinerate the medication.

The label plug 308, 1308 may further comprise a moisture sensor 360 that senses the moisture level inside the label plug (FIGS. 7C, 7D and 10B). The signal of the moisture sensor is transmitted to the communication device 340. If the moisture sensor 360 senses the moisture level is too high due to the presence of spoilage chemical spilled outside the spoilage chemical reservoir, that means the auto-spoilage mechanism may have been deployed and the medications inside the medication cartridge 300, 301, 1300 is not usable. In such a situation, the communication device 340 may override the command to dispense medications sent from the drug dispenser machine 200, 1200, and keep the catching devices 328, 1328a, 1328b covered by the catching device cover 334 so that medications are not dispensed.

The label plug 308, 1308 may come as separated from the carousel 314, 1314. FIGS. 8A and 8B show an example label plug 308 being fitted into a hub 318 of a carousel 314. First, the label plug 308 may be inserted into the hub 318 by aligning the sleeve marker 356 (e.g., an arrow) on the sleeve 326 with a corresponding hub marker 358 (e.g., a corresponding arrow) on the hub 318 of the carousel 314 (FIG. 8A). The sleeve marker may be a projection or a recess. The hub marker may be a corresponding recess or a corresponding projection. Once the sleeve marker 356 is fitted into the corresponding hub marker 358, the sleeve 326 may be pulled out and, as a result, the locking bars 344, the drive arm 330, and the pill cover activating bars 338 are released and engage with the carousel 314 (FIG. 8B).

In some embodiments, the medication cartridge may be premanufactured by a manufacturer, such as a pharmaceutical company or a medication packaging company. The medication cartridge may be packaged by the manufacturer with medication included inside and the label plug has been inserted into the medication cartridge. When a pharmacy fulfills a prescription for a patient, the pharmacy may give the patient the premanufactured medication cartridge, without loading medications into the medication cartridge at the pharmacy. In some embodiments, the pharmacy may assemble a medication cartridge for a patient by loading the medications into a medication cartridge and inserting the label plug into the medication cartridge to secure the medications from being tampered, overdosed, or other improper uses. The manufacturer or the pharmacy may write to the communication device of the medication cartridge with information about the medication loaded inside, such as dosage, dosage frequency, potential adverse reaction with itself or other medications, or the patient's allergy or adverse reaction history. The pharmacy may also write to the communication device with patient-specific information, such as the patient's name and date of birth, to help synchronize the dispensing of medications and reconcile the dispensing logs.

To dispense medications, the drive system 204 of the drug dispenser machine 200, 1200 may engage with the label plug 308, 1308 and, in turn, removes the pill cover 304 away from the pill chamber 316 in the carousel 314, 1314 to dispense the medications in the pill chamber 316. The drug dispenser machine 200, 1200 also communicates with the label plug 308, 1308 to update the dispensing data about the medication cartridge 300, 301, 1300.

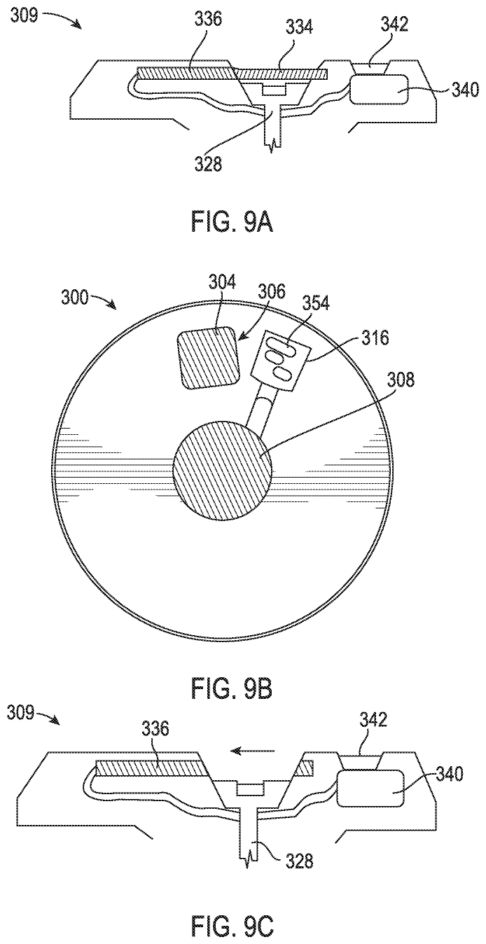

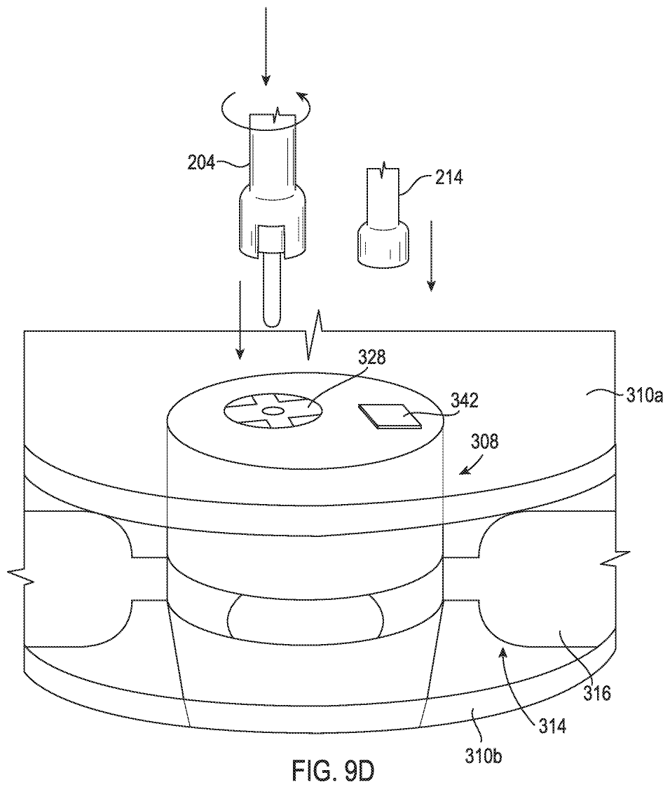

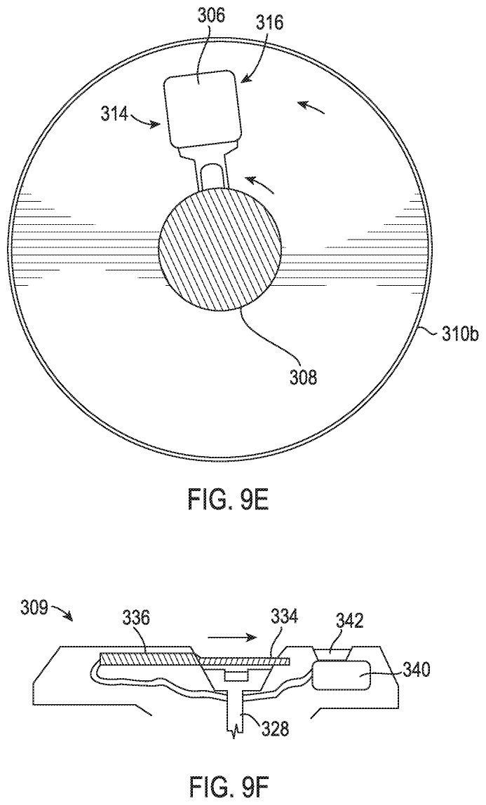

FIGS. 9A-9F show an example process of the drive system 204 engaging with the top portion 309 of the label plug 308 to dispense medications. FIG. 9A shows the top 309 of the label plug 308 at a resting state, where the catching device cover 334 covers the catching device 328. FIG. 9B shows a top view of the medication cartridge 300 at the resting state, where the pill cover 304 covers the pill opening 306 and the pill opening 306 is not aligned with the next pill chamber 316 that holds the medications 354 to be dispensed next. Once the drug dispenser machine 200, 1200 has started a dispensing process, the drug dispenser machine 200, 1200 sends a command to dispense medications to the communication device 340 of the label plug 308. The communication device 340 then sends a command to the cover actuator 336, the cover actuator 336 retracts the catching device cover 334, and the catching device 328 becomes accessible to the drive system 204 (FIG. 9C). The drive system 204 may come into contact and engage with the catching device 328 (FIG. 9D). In some embodiments, the drive system 204 rotates the catching device 328, the label plug 308, the carousel 314, and the pill chamber 316. In some embodiments, the drive system 204 comprises a sear 222, such as a bar or part of a trigger mechanism, which is recessed within the shaft 224 of the drive system 204 when the drive system 204 is not activated, but when the drug dispenser machine activates the auto-spoilage mechanism, the sear emerges from the shaft 224 to engage with the label plug 308 (FIG. 9D) and the auto-spoilage mechanism by collapsing the springs restraining the pins from collapsing the spoilage chemical reservoir. The read/write device 214 may also engage with the communication device 340. In some embodiments, the read/write device 214 engages with the communication device 340 through the read/write device connector 342. The read/write device 214 reads and/or writes dispending data, such as the dispensing log, from and/or to the communication device 340. After the carousel is moved, the pill opening 306 aligns with the pill chamber 316 and, then the pill cover 304 is retracted to allow the medications to be dispensed (FIG. 9E). Once the medications are dispensed, the drive system 204 and the read/write device 214 retract from the label plug 308, the communication device 340 issues a command to the cover actuator 336 to return to the pre-dispensing state, and the cover actuator 336 pushes the catching device cover 334 out to cover the catching device 328 (FIG. 9F). The catching device 328 becomes inaccessible to the drive system 204.

A medication cartridge 1300 may have a multi-layered carousel 1314 (FIGS. 10A-10B). FIG. 10A shows an example medication cartridge 1300 with a carousel 1314 having two layers 1302, 1304. The number of layers is not limited to two as shown in FIG. 10A. Medication cartridges having multiple layers of carousels may be used to dispense medications that last a long duration, e.g., two weeks or longer, without replacing the medication cartridge from the drug dispenser machine.

FIG. 10B shows the cross-section view a multi-layered medication cartridge 1300. The medication cartridge 1300 comprises a multi-layered carousel 1314 and a label plug 1308 driving the carousel 1314. The carousel 1314 may comprise a first layer 1302 and a second layer 1304. The first layer 1302 may be placed on top of the second layer 1304. The first layer 1302 may have the same structure as, or similar structure to, the second layer 1304, sharing the protective plate 310 and the pill cover 304 between the layers. The label plug 1308 may comprise a first catching device 1328a configured to couple with the first layer 1302 of the carousel 1314, and a second catching device 1328b configured to couple with the second layer 1304 of the carousel 1314. The label plug 1308 may comprise a first drive arm 1330a, a second drive arm 1330b, and two or more pill cover activating bar 1338. Once the label plug 1308 is primed by being inserted into the hub 318 and the sleeve 326 being off the label plug 1308, the first drive arm 1330a or the second drive arm engages with the recesses 332 on the pill chambers 316, and the pill cover activating bars 1338 engage with the pill covers 304.

The label plug 1308 may further comprise a spoilage chemical reservoir configured to couple with both layers 1302, 1304 of the carousel 1314. When the label plug 1308 is primed, the channel lip 362 connects the channel 320 leading to the pill chamber 316 with the trough opening 364 of the trough 366 in the label plug 1308. When the auto-spoilage mechanism is deployed, the pins 352 punctures the membrane 368 containing the spoilage chemical 350 and the spoilage chemical 350 flows to the trough 366 and further to the pill chambers 316 through the channels 320 to spoil the medications held in the pill chambers. The pill chamber or the bottom protective plate may comprise a speedbump for the spoilage chemical to have sufficient time to react with the medications in the pill chamber. The speedbump may conform to the shape of the pill chamber outline.

The drive system 204 of a drug dispenser machine 200 may have a variable-depth spindle 1320 (FIGS. 10C and 10D). When the drive system 204 is driving the first layer 1302 of the carousel 1314 to dispense medications held in the first layer 1302, the spindle 1320 of the drive system 204 engages with the first catching device 1328a of the label plug 1308 at a first depth (FIG. 10C). When the drive system 204 is driving the second layer 1304 to dispense medications held in the second layer 1304, the spindle 1320 of the drive system 204 moves to a second depth and engages with the second catching device 1328b of the label plug 1308 (FIG. 10D). In the multi-layered medication cartridge 1300, dispensing medications may start from the second layer 1304, or may start from the first layer 1302 if the pill chamber 316a to be dispensed next in the first layer 1302 will line up with an empty pill chamber 316b in the second layer 1304 during the dispensing process (FIG. 10A).

To increase the number of the layers in a multi-layered medication cartridge, more catching devices may be added below the existing catching devices, or a mirrored label plug may be disposed at the underside of the existing label plug so that the mirrored label plug may be accessed from below.

The drug dispenser system may be for single use, similar to a single-use camera. The pharmacy or the manufacturer delivers one or more medication cartridges within a drug dispenser machine to a user. The single-use drug dispenser system is configured to be difficult for the user to take out the medication cartridge without taking apart the drug dispenser machine. A single-use drug dispenser system offers a cheap and convenient alternative for a user.

A dispenser machine may be used by multiple users. The dispensing logs are coordinated among the users, but individual information may be recorded separately.

The drug dispenser system may comprise a synching terminal 226 (FIG. 1B). In some instances, a patient may have more than one drug dispenser system. The synching terminal 226 may synchronize the drug dispenser systems of the patient to manage all of the patient's medication needs.

Unlike conventional drug dispenser systems, the drug dispenser system disclosed herein conducts a safety check before medications stored in the medication cartridges are dispensed. The safety check comprises checking any potential adverse reaction between doses of the same medication or between different medications, or checking against the user's own history of allergy or adverse medication reactions. If the safety check has failed, the drug dispenser system does not dispense medications. The safety check may comprise an auto-spoilage check, e.g., checking whether the medications have expired or passed the prescribed time. The drug dispenser system may initiate an auto-spoilage mechanism when the auto-spoilage check has failed or when the drug dispenser system is tampered with. The medication cartridges may be returned to the pharmacy. The pharmacy may reuse or refill the medication cartridges. The pharmacy may read the data on the medication cartridge and check the usage compliance of the medication. The safety check and the auto-spoilage mechanism may limit overdose, misuse, or prescription drug abuse problems that have caused a public health crisis in the U.S.

This disclosure, its aspects and implementations, are not limited to the specific components or assembly procedures disclosed herein. Many additional components and assembly procedures known in the art consistent with the intended drug dispenser systems and methods of dispensing medications will become apparent for use with implementations of the apparatus and methods in this disclosure. In places where the description above refers to particular implementations of drug dispenser systems, it should be readily apparent that a number of modifications may be made without departing from the spirit thereof and that these implementations may be applied to other drug dispenser systems. The presently disclosed implementations are, therefore, to be considered in all respects as illustrative and not restrictive, the scope of the disclosure being indicated by the appended claims rather than the foregoing description. All changes that come within the meaning of, and range of equivalency of, the description are intended to be embraced therein. Accordingly, for example, although particular drug dispenser systems and methods of dispensing medications are disclosed, such apparatus, methods, and implementing components may comprise any shape, size, style, type, model, version, class, grade, measurement, concentration, material, quantity, or the like as is known in the art for such apparatus, methods, and implementing components, and/or the like consistent with the intended operation of the drug dispenser systems and methods of dispensing medications may be used.

1. A drug dispenser system comprising: a drug dispenser machine comprising:
a controller having a processor and a memory, the controller configured to conduct a safety check,
a user interface configured to interact with a user,
a drive system configured to couple to the controller, and
a display configured to display information sent from the controller;
a medication cartridge releasably loaded onto the drug dispenser machine, the medication cartridge including:
at least one pill chamber,
at least one protective plate covering at least a majority of the at least one pill chamber, and
a label plug configured to couple to the at least one pill chamber; and
wherein the drive system of the drug dispenser machine is configured to move the label plug and, in turn, move the at least one pill chamber upon receipt of a command to dispense a medication sent from the controller when the safety check is passed.

2. The drug dispenser system of claim 1, wherein the safety check comprises at least one of checking whether a minimum period of time between doses has elapsed, checking whether a request time of the safety check is outside an adversely indicated window of another medication, and checking whether a user's medication reaction history indicates the medication is safe for the user.

3. The drug dispenser system of claim 1, wherein: the drug dispenser machine further comprises a read/write device configured to couple to the controller; the label plug of the medication cartridge further comprises a communication device; and the read/write device and the communication device are configured to communicate with each other.

4. The drug dispenser system of claim 1, further comprising an indicator configured to couple to the controller, wherein the controller is configured to send a command to set the indicator into a warning mode when the safety check is not passed.

5. The drug dispenser system of claim 1, wherein the medication cartridge comprises a substantially circular foot print, the medication cartridge further comprising a carousel containing the at least one pill chamber.

6. The drug dispenser system of claim 5, further comprising a first medication cartridge and a second medication cartridge, wherein:
the first medication cartridge comprises a first medication;
the second medication cartridge comprises a second medication;
the drug dispenser machine further comprises a turntable; and
the first medication cartridge and the second medication cartridge are disposed on the turntable.

7. The drug dispenser system of claim 1, further comprising a first layer of the medication cartridge and a second layer of the medication cartridge, the first layer disposed over the second layer.

8. The drug dispenser system of claim 1, wherein the label plug of the medication cartridge further comprises a spoilage chemical reservoir, the label plug configured to deploy a spoilage chemical stored in the spoilage chemical reservoir into each of the at least one pill chamber of the medication cartridge when an auto-spoilage condition is met.

9. The drug dispenser system of claim 1, wherein the medication cartridge is packaged by a manufacturer with a medication inside the at least one pill chamber, and the label plug of the medication cartridge is primed and inserted into the medication cartridge by the manufacturer.

10. A drug dispenser system comprising:
a drug dispenser machine comprising:
a controller comprising a processor and a memory,
a drive system configured to couple to the controller, and
a display configured to display information sent from the controller; and
wherein the drug dispenser machine is configured to receive a medication cartridge, the controller of the drug dispenser machine is configured to conduct a safety check upon receipt of a dispensing request from a user, and the drive system is configured to move the medication cartridge upon receipt of a command to dispense a medication from the controller when the safety check is passed.

11. The drug dispenser system of claim 10, further comprising one or more medication cartridges releasably loaded onto the drug dispenser machine, the one or more medication cartridges comprising at least one pill chamber.

12. The drug dispenser system of claim 11, wherein the one or more medication cartridges further comprises a spoilage chemical reservoir, the drug dispenser machine configured to deploy spoilage chemical stored in the spoilage chemical reservoir into each of the at least one pill chamber of the one or more medication cartridges when an auto-spoilage condition is met.

13. The drug dispenser system of claim 10, wherein the safety check comprises at least one of:
checking whether a minimum period of time between doses has elapsed;
checking whether a request time of the safety check is outside an adversely-indicated window of another medication; and
checking whether the user's medication reaction history indicates the medication is safe for the user.

14. A drug dispenser system, comprising:
receiving a request, by a controller, of dispensing a new dose of a medication for a user, wherein at least one dose of the medication is held inside a drug dispenser machine;
conducting, by the controller, a safety check;
indicating, by a display, whether the new dose of the medication is safe to be dispensed; and
dispensing the medication from the drug dispenser machine if the safety check is passed.

15. The drug dispenser system of claim 14, wherein the safety check comprises checking whether a minimum period of time between the new dose and an immediately-prior dose of the medication has elapsed or whether a request time of the safety check is outside an adversely-indicated window of another medication.

16. The drug dispenser system of claim 15, wherein the drug dispenser system is configured to display an amount of time to wait before the new dose of the medication is dispensed if the minimum period of time between the new dose and the immediately-prior dose has not elapsed or if the request time of the safety check is within the adversely-indicated window of another medication.

17. The drug dispenser system of claim 14, wherein the safety check comprises checking whether the user's medication reaction history indicates the medication is safe for the user.

18. The drug dispenser system of claim 17, wherein the drug dispenser system is configured to set an indicator to a warning mode and display at least a portion of the user's medication reaction history that indicates the medication is unsafe for the user.

19. The drug dispenser system of claim 14, wherein the drug dispenser system is configured to check whether the medication has expired or whether a request time of the safety check is after a prescribed time of the medication.

20. The drug dispenser system of claim 19, wherein the drug dispenser system is configured to start an auto-spoilage mechanism if the medication has expired or if the request time of the safety check is after the prescribed time of the medication.
